(19) United States
(12) Patent Application Publication
(10) Pub. No.: US 2026/0364572 A1
(43) Pub. Date: Nov. 19, 2026

(54) CATALYST FOR PRODUCING PROPYLENE GLYCOL BY HYDROCRACKING REACTION OF GLYCEROL AND METHOD FOR PRODUCING SAME

(71) Applicant: KOREA RESEARCH INSTITUTE OF CHEMICAL TECHNOLOGY, Daejeon (KR)

(72) Inventors: Dong Won HWANG, Daejeon (KR); Seung Hyeok CHA, Daejeon (KR); Hae Rin LEE, Daejeon (KR); Ma Eum LEE, Daejeon (KR)

(73) Assignee: KOREA RESEARCH INSTITUTE OF CHEMICAL TECHNOLOGY, Daejeon (KR)

(21) Appl. No.: 19/722,094

(22) Filed: Jun. 26, 2026

Related U.S. Application Data
(63) Continuation of application No. PCT/KR2024/020300, filed on Dec. 13, 2024.

(30) Foreign Application Priority Data
Feb. 2, 2024 (KR) 10-2024-0016684

(51) Int. Cl. B01J 23/78; B01J 35/30; B01J 35/40; B01J 35/45; B01J 37/00; B01J 37/02; B01J 37/03; B01J 37/04; B01J 37/06; B01J 37/08; B01J 37/18; C07C 29/60

(52) U.S. Cl. CPC B01J 23/78; B01J 35/394; B01J 35/40; B01J 35/45; B01J 37/009; B01J 37/0236; B01J 37/031; B01J 37/04; B01J 37/06; B01J 37/08; B01J 37/18; C07C 29/60

(57) ABSTRACT

The present invention relates to: a nanocomposite catalyst and a method for producing same, wherein, in producing propylene glycol from glycerol through a hydrocracking reaction using a catalyst, the catalyst is K₂O—CuO—SiO₂ consisting of K oxide (K₂O), Cu oxide (CuO), and silica (SiO₂); and a method for producing propylene glycol from glycerol through a hydrocracking reaction using the catalyst.

### CROSS REFERENCE TO RELATED APPLICATIONS

The present application is a continuation of PCT International Application No. PCT/KR2024/020300, which has an International filing date of Dec. 13, 2024, and which claims priority to Korean Patent Application No. 10-2024-0016684, filed on Feb. 2, 2024, the entire contents of each of which is incorporated herein for all purposes by this reference.

### TECHNICAL FIELD

The present disclosure relates generally to a catalyst for producing propylene glycol by a hydrogenolysis reaction of glycerol and a method for producing the same. More specifically, the disclosure relates to a K₂O—CuO—SiO₂ nanocomposite catalyst exhibiting excellent glycerol conversion and propylene glycol selectivity in the production of propylene glycol through a hydrogenolysis reaction of glycerol, and to a method for producing the same.

### BACKGROUND ART

The widespread use on fossil fuels in modern society inevitably has given rise to climate and environmental challenges, including global warming, thereby driving sustained interest in and demand for carbon neutrality. As one representative approach toward achieving carbon neutrality, partially replacing fossil fuels currently used as energy sources with biomass-derived biodiesel has been proposed. It is widely considered an environmentally sustainable strategy, particularly in view of the effective utilization of abundant biomass resources available in nature.

In the case of biodiesel produced through the transesterification of triglycerides contained in the biomass, a significant amount of glycerol is generated as a byproduct, typically in an amount of approximately 10 to 100 kg per ton of biodiesel produced. Accordingly, considerable efforts have been made to identify practical applications for glycerol in order to pursue its valorization. Since the 1990s, glycerol has been utilized in the cosmetics, pharmaceutical, and food industries as a humectant, excipient, and additive. Despite satisfying demand in these sectors, the increase in biodiesel production has ultimately led to an oversupply of glycerol, thereby necessitating the exploration of alternative industrial applications, such as high-value chemical feedstocks, polymer building blocks, and fuel additives.

Meanwhile, propylene glycol (PG), also known as 1,2-propanediol, is a critical industrial chemical with substantial demand in a wide range of applications, including polyester resins, polyurethanes, cosmetics, and deicing additives. Traditionally, propylene glycol production has primarily relied on petroleum-derived propylene oxide via chlorohydrin or hydroperoxide routes. However, these conventional processes suffer from significant drawbacks, including the use of toxic oxidizing agents and the generation of chlorinated byproducts. Consequently, hydrogenolysis of glycerol has emerged as a promising alternative route for the sustainable production of propylene glycol.

Propylene glycol produced through a hydrogenolysis reaction of glycerol is recognized as a high-value-added chemical with broad applicability, serving as an intermediate in pharmaceutical and cosmetic formulations, a precursor for polyester and unsaturated polyester synthesis, and a plasticizer for cellophane.

However, there has not been much research on catalysts for the above reaction. For example, Korean Patent Application Publication No. 10-2011-0116480 A discloses the use of a noble metal catalyst supported on a hydrotalcite-like material, which exhibits a glycerol conversion of only 58.5% and a propylene glycol selectivity of 85.5% under reaction conditions of 180 °C, 40 bar, and continuous hydrogen flow, indicating insufficient catalytic performance in terms of both conversion and selectivity. Additionally, in U.S. Pat. No. 5,616,817 A, a mixed catalyst of cobalt, copper, manganese, and molybdenum is produced and used for the conversion of glycerol into propylene glycol. However, the disclosed reaction conditions require extremely harsh operating parameters, including pressures of 100 to 700 bar and temperatures of up to 270 °C.

More recently, copper-chromium catalysts with a spinel structure have been reported as potential catalysts for glycerol hydrogenolysis. However, such catalysts generally exhibit catalytic activity only under elevated hydrogen pressures exceeding 80 bar, which imposes a substantial burden on chemical process operation and presents significant challenges for commercial production of propylene glycol.

Meanwhile, Korean Patent Application Publication No. 10-2015-0125422 A discloses a NiO—CuO/SiO₂ nanocomposite catalyst produced via a sol-gel process using a nickel precursor compound, a copper precursor compound, and SiO₂. Although this catalyst demonstrates relatively high glycerol-to-propylene glycol conversion efficiency under comparatively low hydrogen pressure conditions, it still exhibits limited long-term stability.

Therefore, there exists a strong need for the development of a catalyst capable of achieving high glycerol conversion and high propylene glycol selectivity under relatively low hydrogen pressure and mild temperature conditions, while simultaneously ensuring excellent long-term catalytic stability.

### DOCUMENTS OF RELATED ART

### [Patent Documents]

(Patent Document 1) Korean Patent Application Publication No. 10-2011-0116480 (Published on Oct. 26, 2011)
(Patent Document 2) U.S. Pat. No. 5,616,817 (Registered on Apr. 1, 1997)
(Patent Document 3) Korean Patent Application Publication No. 10-2015-0125422 (Published on Nov. 9, 2015)

### DISCLOSURE

### Technical Problem

Accordingly, the present disclosure has been made keeping in mind the above problems occurring in the related art, the present disclosure aims to provide a catalyst and a method for producing the same, in which, in producing propylene glycol from glycerol through a hydrogenolysis reaction in the presence of a catalyst, the catalyst enables stable production of propylene glycol over an extended period even under relatively low hydrogen pressure and low reaction temperature conditions.

Additionally, the present disclosure aims to provide a method for producing propylene glycol from glycerol in the presence of the above catalyst.

### Technical Solution

The present disclosure provides a catalyst for producing propylene glycol by a hydrogenolysis reaction of glycerol, the catalyst including a K oxide (K₂O), a Cu oxide (CuO), and silica (SiO₂).

In the catalyst, the K oxide (K₂O) and Cu oxide (CuO) may be presented in a total amount of 50 to 95 wt %, based on the total weight of the catalyst, the K oxide (K₂O) may be presented in an amount of 0.1 to 10.0 wt %, based on the total weight of the catalyst, and the silica (SiO₂) may have a particle size of 5 nm to 10 μm.

Additionally, the present disclosure provides a method for producing a catalyst for producing propylene glycol by a hydrogenolysis reaction of glycerol, the method including: (a) preparing a reaction mixture by adding a silica sol, a K₂O precursor, and a CuO precursor to a solvent, followed by mixing and reacting the components; (b) forming a precipitate by adjusting a pH of the reaction mixture prepared in step (a), followed by aging the precipitate; and (c) producing a K₂O—CuO—SiO₂ nanocomposite catalyst by washing, filtering, drying, and calcining the precipitate aged in step (b).

In one embodiment according to the method of the present disclosure, in step a), the silica sol, the K₂O precursor, and the CuO precursor may be added to the solvent such that SiO₂ is presented in an amount of 5 to 50 wt %, K₂O is presented in an amount of 0.1 to 10 wt %, and a Cu/K molar ratio is 3 to 400 in the finally produced K₂O—CuO—SiO₂ nanocomposite catalyst.

Additionally, the present disclosure provides a method for producing propylene glycol by a hydrogenolysis reaction of glycerol, the method including: a) preparing an aqueous glycerol solution; b) charging the catalyst into a reactor and reducing the catalyst using hydrogen gas; and c) producing propylene glycol by introducing the aqueous glycerol solution together with hydrogen gas into the reactor and allowing them to react.

In the method for producing propylene glycol according to the present disclosure, in step c), a hydrogen/glycerol molar ratio may be 2 to 200, a reaction temperature may be 180 °C to 260 °C, a reaction pressure may be 15 to 45 bar, and a glycerol feed rate may be 0.05 to 3 g/h per 1 g of catalyst.

### Advantageous Effects

When propylene glycol is produced by a hydrogenolysis reaction of glycerol in the presence of a K₂O—CuO—SiO₂ nanocomposite catalyst according to the present disclosure, high glycerol conversion and high propylene glycol selectivity can be achieved under relatively low hydrogen pressure and low reaction temperature conditions. Also, the K₂O—CuO—SiO₂ nanocomposite catalyst can exhibit excellent long-term catalytic stability, thereby enabling the production of propylene glycol from glycerol at high yield over an extended period through the hydrogenolysis reaction.

### BEST MODE

Hereinafter, a catalyst for producing propylene glycol by a hydrogenolysis reaction of glycerol, a method for producing the same, and a method for producing propylene glycol in the presence of the catalyst will be described in detail such that the disclosure can be easily embodied by one of ordinary skill in the art to which this disclosure belongs.

In the following description of preferred embodiments of the present disclosure, detailed descriptions of known functions and components incorporated herein will be omitted when it may make the subject matter of the present disclosure unclear.

Furthermore, the description proposed herein is just a preferable example for the purpose of illustration only, not intended to limit the scope of the disclosure, so it should be understood that other equivalents and modifications could be made thereto without departing from the scope of the disclosure.

The present disclosure relates to a K₂O—CuO—SiO₂ nanocomposite catalyst and a method for producing the same, in which, in producing propylene glycol from glycerol through a hydrogenolysis reaction in the presence of a catalyst, the catalyst enables stable production of propylene glycol over an extended period even under relatively low hydrogen pressure and low reaction temperature conditions. The present disclosure also relates to a method for producing propylene glycol by subjecting glycerol to a hydrogenolysis reaction in the presence of the K₂O—CuO—SiO₂ nanocomposite catalyst.

Hereinafter, a detailed description will be given of a catalyst for producing propylene glycol by a hydrogenolysis reaction of glycerol according to the present disclosure and a method for producing the same.

A catalyst for producing propylene glycol by a hydrogenation reaction of glycerol according to one embodiment of the present disclosure is a nanocomposite catalyst consisting of a K component, a Cu component, and silica, which may be expressed as K—Cu/SiO₂, more specifically, K₂O—CuO—SiO₂.

The nanocomposite catalyst according to the present disclosure may exist in a form in which K, Cu, and Si components are present as a mixture of a complex oxide and a mixed oxide. For example, the K and Cu components may form a complex oxide represented as K₂O—CuO and exist in a form supported on SiO₂ particles or as separate particles. At this time, it is preferable that the SiO₂ has a particle size of 5 nm to 10 μm. When the particle size of the silica falls within the above-defined range, glycerol conversion and propylene glycol selectivity in the hydrogenolysis reaction of glycerol are improved.

In the K₂O—CuO—SiO₂ nanocomposite catalyst according to the present disclosure, when the contents of the (K+Cu) component and the Si component are expressed on an oxide weight basis, the (K+Cu) component may be present in an amount of 50 to 95 wt %, preferably 60 to 90 wt %, and more preferably 70 to 80 wt %, based on the total weight of the catalyst, while SiO₂ may be presented in an amount of 5 to 50 wt %, preferably 10 to 40 wt %, and more preferably 20 to 30 wt %.

Additionally, in the K₂O—CuO—SiO₂ nanocomposite catalyst according to the present disclosure, the K component is used in a relatively small amount, and the K component may be presented in an amount of 0.1 to 10 wt %, preferably 1 to 5 wt %, and more preferably 2.5 to 3.5 wt %, based on the total weight of the catalyst on an oxide weight basis.

Additionally, in the present disclosure, the Cu/K molar ratio, i.e., the molar ratio of the Cu component to the K component, may be 3 to 400, preferably 5 to 40. When the Cu/K molar ratio is less than 3, glycerol conversion may be undesirably low, whereas when the Cu/K molar ratio exceeds 400, the catalyst may exhibit poor stability.

As the K content falls within the more preferred range, the hydrogen adsorption capability attributable to K and the hydrogen spillover effect are enhanced, thereby improving glycerol conversion and propylene glycol selectivity.

Additionally, the K₂O—CuO—SiO₂ nanocomposite catalyst according to the present disclosure may be processed into a catalyst molded body. Although the shape of the catalyst molded body is not particularly limited, it may be in the form of powder or pellet. In particular, since a pellet form is preferable for application in a continuous process, the catalyst may be produced and used in pellet form according to methods commonly employed in the art.

The K₂O—CuO—SiO₂ nanocomposite catalyst according to the present disclosure may be used after pre-treatment under a hydrogen gas flow during the hydrogenolysis reaction of glycerol.

Additionally, the K₂O—CuO—SiO₂ nanocomposite catalyst according to the present disclosure is produced by a co-precipitation method using a K₂O precursor, a CuO precursor, and SiO₂ particles.

More specifically, a method for producing the K₂O—CuO—SiO₂ nanocomposite catalyst according to the present disclosure includes: (a) preparing a reaction mixture by adding a silica sol, a K₂O precursor, and a CuO precursor to a solvent, followed by mixing and reacting the components; (b) forming a precipitate by adjusting the pH of the reaction mixture prepared in step (a), followed by aging the precipitate; and (c) producing a catalyst powder by washing, filtering, drying, and calcining the precipitate aged in step (b). The catalyst powder (i.e., the calcined precipitate) produced through the above steps may be molded by a process involving adding an organic or inorganic binder thereto, kneading the resulting mixture, and then subjecting the kneaded mixture to extrusion molding.

The solvent used in step a) is not particularly limited, as long as it is capable of dissolving the precursors, and may be selected from polar or nonpolar solvents. For example, a lower alcohol such as ethanol or butanol, water, or a mixture thereof may be used.

Additionally, the K₂O precursor and the CuO precursor used in step a) are not particularly limited, but may selected, alone or in combination of two or more, from the group consisting of carbides, oxides, chlorides, nitrides, sulfides, sulfates, nitrates, and hydrates or anhydrides thereof.

Additionally, in step a), in adding the silica sol, the K₂O precursor, and the CuO precursor to the solvent, the silica sol may be added such that SiO₂ is presented in an amount of 5 to 50 wt %, preferably 10 to 40 wt %, and more preferably 20 to 30 wt % in the finally produced K₂O—CuO—SiO₂ nanocomposite catalyst.

Additionally, the K₂O precursor is added such that K₂O is presented in an amount of 0.1 to 10 wt %, preferably 1 to 5 wt %, and more preferably 2.5 to 3.5 wt % in the finally produced K₂O—CuO—SiO₂ nanocomposite catalyst.

Additionally, the CuO precursor is added such that the Cu/K molar ratio is 3 to 400, preferably 5 to 40 in the finally produced K₂O—CuO—SiO₂ nanocomposite catalyst.

The adjusting of the pH of the reaction mixture in step b) may be carried out by adding an aqueous alkali carbonate solution or aqueous sodium hydroxide solution to the metal precursor solution. The specific pH value may vary within the range of 7 to 13 depending on the content of the metal components contained in the metal precursor solution.

Additionally, the particle size and formation rate of the precipitate may vary depending on the concentration and addition rate of the aqueous alkali carbonate solution or aqueous sodium hydroxide solution used in step b). For example, when the addition rate of the aqueous alkali carbonate solution or aqueous sodium hydroxide solution is excessively fast, or when the concentration thereof is excessively high, the resulting precipitate particles may become excessively large, thereby causing a reduction in catalytic activity. Whereas, when the addition rate is excessively slow or the concentration is excessively low, the efficiency of catalyst production may be reduced. Therefore, in order to improve catalytic activity and enhance the efficiency of catalyst production, the concentration of the aqueous alkali carbonate solution or aqueous sodium hydroxide solution may be controlled within the range of 0.5 to 3 mol/L, and the aqueous alkali carbonate solution or aqueous sodium hydroxide solution may be added to the metal precursor solution at a rate of 0.1 to 10 mL/min.

Additionally, in step (b), the precipitate may be subjected to aging once the pH of the reaction mixture reaches a specific value within the range of 9 to 13. The aging may be performed at 50 °C to 90 °C for 2 to 12 hours to allow the precipitation reaction to proceed sufficiently. When the aging temperature is excessively low or excessively high, it may be difficult to form a precipitate having a particle size capable of providing high catalytic activity. Therefore, as described above, the aging temperature may be set within the range of 50 °C to 100 °C, preferably 60 °C to 80 °C.

Step (c) is a process for sufficiently washing the precipitate with distilled water until the aqueous alkali carbonate solution or aqueous sodium hydroxide solution remaining in the precipitate is completely removed. After the washing step, the washed precipitate is filtered and dried. The drying is sufficiently carried out at 80 °C to 200 °C until the precipitate is completely dried.

Additionally, in step (c), the dried precipitate is subjected to calcination to remove residual organic matter remaining in the precipitate and to form an appropriate alloy phase. The calcination may be carried out within a temperature range of 400 °C to 700 °C under an air atmosphere or an oxygen atmosphere, and preferably at 500 °C to 600 °C, under which a suitable alloy phase may be formed. The specific calcination temperature may be determined as a temperature capable of forming an alloy phase exhibiting high catalytic activity, and may vary depending on the content of the metal components contained in the formed precipitate.

Additionally, a solid catalyst may be manufactured by adding an inorganic or organic binder to the catalyst powder produced in step (c), kneading the resulting mixture, and then subjecting the kneaded mixture to extrusion molding. The inorganic binder may be selected from other inorganic oxides, such as silica, zirconia, and titania, while the organic binder preferably includes one or more selected from ceramic organic binders containing ether linkages. Examples of the organic binder include, but are not limited to, PMB-15U, PMB-40H, and MC-40H.

The catalyst powder to which the binder has been added is uniformly mixed using a mixer, followed by addition of distilled water to prepare the mixture in a kneaded form suitable for molding. The resulting kneaded mixture is then extruded through an extruder under an appropriate pressure and cut at regular intervals to produce a pellet-type catalyst. The pellet-type catalyst thus produced is subsequently subjected to a drying step to remove moisture.

Meanwhile, when the organic binder is used for extrusion molding, the dried catalyst may be further subjected to calcination at a high temperature in order to remove the organic binder, which may interfere with catalytic activity, and to improve the mechanical strength of the catalyst. At this time, the calcination temperature is preferably about 550 °C and the calcination time is preferably 6 to 8 hours.

Additionally, a method for producing propylene glycol by a hydrogenolysis reaction of glycerol according to another embodiment of the present disclosure includes: i) preparing an aqueous glycerol solution; ii) charging a catalyst into a reactor and reducing the catalyst using hydrogen gas; and iii) producing propylene glycol by introducing the aqueous glycerol solution together with hydrogen gas into the reactor and allowing them to react.

In step i), when the aqueous glycerol solution contains non-volatile impurities, such as salts generated during biodiesel synthesis, it is preferable, in terms of preventing catalyst deactivation, to first separate the glycerol using an evaporator and then supply the separated glycerol to a catalytic reactor. However, in the case of impurities (methanol, water, etc.) having boiling points lower than that of glycerol, such impurities may be supplied to the catalytic reactor together with the glycerol. The concentration of glycerol ultimately introduced into the catalytic reactor may generally be selected within the range of 10 to 98%, preferably 20 to 95%, and more preferably 30 to 90%.

Additionally, in step ii), the catalyst is reduced at a temperature of 250 °C to 350 °C, preferably 270 °C to 330 °C, during catalytic reduction.

Additionally, in step iii), the hydrogen/glycerol molar ratio is 2 to 200, preferably 3 to 100, more preferably 4 to 30, and even more preferably 5 to 25.

Additionally, in step iii), the reaction temperature is 180 °C to 260 °C, preferably 200 °C to 240 °C and more preferably 210 °C to 230 °C, and the reaction pressure is 15 to 45 bar, preferably 20 to 40 bar.

Additionally, in step iii), the glycerol feed rate is 0.05 to 10 g/h per 1 g of catalyst, preferably 0.1 to 8 g/h, and more preferably 0.2 to 5 g/h.

Meanwhile, the method for producing propylene glycol by the hydrogenolysis reaction of glycerol according to the embodiment of the present disclosure may further include: separating propylene glycol from glycerol and water in the reaction product obtained from step (iii) by a method such as distillation or extraction.

At this time, when the catalyst of the present disclosure is molded into a predetermined form, such as pellets, and used, it may facilitate continuous operation of the above production method.

One of the features of the present disclosure is that the temperature and pressure of the hydrogenolysis reaction are appropriately controlled so as to maintain glycerol in a liquid phase and bring the same into contact with the catalyst and hydrogen, thereby enabling the catalytic hydrogenolysis reaction to be carried out in the liquid phase. Therefore, disadvantages or issues associated with gas-phase reactions are avoided, and problems such as coking, which are inevitably accompanied in gas-phase catalytic reactions, are substantially mitigated.

Hereinafter, the effects of the catalyst for producing propylene glycol according to the present disclosure in producing propylene glycol through the hydrogenolysis reaction of glycerol in the presence of the catalyst will be examined through examples. The following examples are provided to illustrate one or more preferred embodiments of the disclosure, but the disclosure is not limited to those embodiments. A number of changes may be made to the following examples without departing from the scope of the present disclosure.

### Example 1: Glycerol Hydrogenolysis Reaction in the Presence of K₂O(3)-CuO(77)-SiO₂ Nanocomposite Catalyst

In 500 mL of distilled water, 3.56 g of colloidal silica sol (Ludox SM-30, 30 wt %), 12.50 g of Cu(NO₃)₂·3H₂O, and 0.23 g of KNO₃·H₂O were prepared. While maintaining the temperature at 5 °C under stirring at 600 rpm, the silica sol and the respective aqueous metal precursor solutions were simultaneously and slowly added at a rate of 1 to 6 cc/min and mixed together. Subsequently, 1 M NaOH was slowly added under stirring until the pH of the reaction mixture reached 9.2, followed by reaction under stirring at room temperature for 18 hours. The resulting mixture was then aged at 70 °C for 3 hours to form a precipitate.

The reaction mixture was cooled to room temperature and filtered to recover the precipitate. The recovered precipitate was washed with 1000 mL of distilled water until Na ions were no longer detected, and then dried at 100 °C for 12 hours to obtain the precipitate in powder form.

The obtained powder was calcined at 550 °C in an air atmosphere for 6 hours to produce a K₂O(3)-CuO(77)-SiO₂ nanocomposite catalyst.

1.0 g of the K₂O(3)-CuO(77)-SiO₂ nanocomposite catalyst produced in the above step was charged into a fixed-bed reactor and treated with 10% H₂ gas at 290 °C for 5 hours. Thereafter, an 80 wt % aqueous glycerol solution was supplied at a weight hourly space velocity (WHSV) of 0.5 h⁻¹, while hydrogen was simultaneously supplied at a flow rate of 48 cc/min. At this time, the reactor temperature was maintained at 220 °C, and the pressure was maintained at 30 bar. Under these conditions, the reaction was carried out for 342 hours, after which the reaction products were analyzed by gas chromatography. The results are summarized in Table 1 below.

### Example 2: Glycerol Hydrogenolysis Reaction in the Presence of K₂O(1)-CuO(79)-SiO₂ Nanocomposite Catalyst

A nanocomposite catalyst was produced in the same manner as in Example 1, except that 3.56 g of silica sol (Ludox SM-30, 30 wt %), 12.50 g of Cu(NO₃)₂·3H₂O, and 0.07 g of KNO₃·H₂C were used.

The hydrogenolysis reaction of glycerol was carried out under the same method and conditions as those described in Example 1, and the resulting products were analyzed by gas chromatography. The results are summarized in Table 1 below.

### Example 3: Glycerol Hydrogenolysis Reaction in the Presence of K₂O(5)-CuO(75)-SiO₂ Nanocomposite Catalyst

A nanocomposite catalyst was produced in the same manner as in Example 1, except that 3.56 g of silica sol (Ludox SM-30, 30 wt %), 12.50 g of Cu(NOS)₂·3H₂O, and 0.41 g of KNO₃·H₂C were used.

The hydrogenolysis reaction of glycerol was carried out under the same method and conditions as those described in Example 1, and the resulting products were analyzed by gas chromatography. The results are summarized shown in Table 1 below.

### Comparative Example 1: Glycerol Hydrogenolysis Reaction in the Presence of CuO(10)-SiO₂ Nanocomposite Catalyst

A nanocomposite catalyst was produced in the same manner as in Example 1, except that no K precursor was used, and 1.56 g of Cu(NO₃)₂·3H₂O and 3.43 g of colloidal silica were used.

The hydrogenolysis reaction of glycerol was carried out under the same method and conditions as those described in Example 1, and the resulting products were analyzed by gas chromatography. The results are summarized in Table 1 below.

### Comparative Example 2: Glycerol Hydrogenolysis Reaction in the Presence of CuO(80)-SiO₂ Nanocomposite Catalyst

A nanocomposite catalyst was produced in the same manner as in Example 1, except that no K precursor was used, and 12.50 g of Cu(NO₃)₂·3H₂O and 3.43 g of colloidal silica were used.

The hydrogenolysis reaction of glycerol was carried out under the same method and conditions as those described in Example 1, and the resulting products were analyzed by gas chromatography. The results are summarized in Table 1 below.

TABLE 1

| | | Reaction time | | | |
|---|---|---|---|---|---|
| | | 0~26 h | | 374~398 h | |
| | | Glycerol | PG | Glycerol | PG |
| | | conversion | selectivity | conversion | selectivity |
| Examples | Catalysts | (%) | (%) | (%) | (%) |
| Example 1 | K₂O(3)—CuO(77)—SiO₂ | 99.90 | 93.09 | 97.80 | 97.65 |
| Example 2 | K₂O(1)—CuO(79)—SiO₂ | 99.9 | 92.03 | 49.25 | 96.32 |
| Example 3 | K₂O(5)—CuO(75)—SiO₂ | 99.5 | 94.45 | 69.17 | 96.80 |
| Comparative | CuO(10)—SiO₂ | 61.25 | 71.56 | 41.21 | 68.12 |
| Example 1 | | | | | |
| Comparative | CuO(80)—SiO₂ | 95.5 | 96.42 | 42.36 | 71.47 |
| Example 2 | | | | | |

As shown in Table 1, it can be confirmed that Examples 1 to 3, which further contain K, exhibited superior glycerol conversion and selectivity, and the glycerol conversion and selectivity were maintained without significant decrease even during prolonged reaction operation.

Although the present disclosure has been described above with reference to the embodiments described herein or shown in the accompanying drawings, these embodiments are merely illustrative. Those skilled in the art will appreciate that various modifications, additions, and substitutions are possible. Therefore, the scope of protection of the present disclosure should be defined by the appended claims.

### INDUSTRIAL APPLICABILITY

When propylene glycol is produced by a hydrogenolysis reaction of glycerol in the presence of a K₂O—CuO—SiO₂ nanocomposite catalyst according to the present disclosure, high glycerol conversion and high propylene glycol selectivity can be achieved under relatively low hydrogen pressure and low reaction temperature conditions. Also, the K₂O—CuO—SiO₂ nanocomposite catalyst can exhibit excellent catalytic stability, thereby enabling the production of propylene glycol from glycerol at high yield over an extended period through the hydrogenolysis reaction. Accordingly, the present disclosure can find a wide application in industrial fields requiring propylene glycol, including polyester resins, polyurethanes, cosmetics, and deicing additives.

1. A catalyst for producing propylene glycol by a hydrogenolysis reaction of glycerol, the catalyst comprising:
a K oxide (K₂O);
a Cu oxide (CuO); and
silica (SiO₂).

2. The catalyst of claim 1, wherein the K oxide (K₂O) and the Cu oxide (CuO) are presented in a total amount of 50 to 95 wt %, based on the total weight of the catalyst.

3. The catalyst of claim 2, wherein the K oxide (K₂O) is presented in an amount of 0.1 to 10.0 wt %, based on the total weight of the catalyst.

4. The catalyst of claim 1, wherein the silica (SiO₂) has a particle size of 5 nm to 10 μm.

5. A method for producing a catalyst for producing propylene glycol by a hydrogenolysis reaction of glycerol, the method comprising:
(a) preparing a reaction mixture by adding a silica sol, a K₂O precursor, and a CuO precursor to a solvent, followed by mixing and reacting the components;
(b) forming a precipitate by adjusting a pH of the reaction mixture prepared in step (a), followed by aging the precipitate; and
(c) producing a K₂O—CuO—SiO₂ nanocomposite catalyst by washing, filtering, drying, and calcining the precipitate aged in step (b).

6. The method of claim 5, wherein in step a), the silica sol, the K₂O precursor, and the CuO precursor are added to the solvent such that SiO₂ is presented in an amount of 5 to 50 wt %, K₂O is presented in an amount of 0.1 to 10 wt %, and a Cu/K molar ratio is 3 to 400 in the finally produced K₂O—CuO—SiO₂ nanocomposite catalyst.

7. A method for producing propylene glycol by a hydrogenolysis reaction of glycerol in the presence of the catalyst of claim 1, the method comprising:
a) preparing an aqueous glycerol solution;
b) charging the catalyst into a reactor and reducing the catalyst using hydrogen gas; and
c) producing propylene glycol by introducing the aqueous glycerol solution together with hydrogen gas into the reactor and allowing them to react.

8. The method of claim 7, wherein in step c), a hydrogen/glycerol molar ratio is 2 to 200.

9. The method of claim 7, wherein in step c), a reaction temperature is 180 °C to 260 °C and a reaction pressure is 15 to 45 bar.

10. The method of claim 7, wherein in step c), a glycerol feed rate is 0.05 to 3 g/h per 1 g of catalyst.

11. A method for producing propylene glycol by a hydrogenolysis reaction of glycerol in the presence of the catalyst produced according to the method of claim 5, the method comprising:
a) preparing an aqueous glycerol solution;
b) charging the catalyst into a reactor and reducing the catalyst using hydrogen gas; and
c) producing propylene glycol by introducing the aqueous glycerol solution together with hydrogen gas into the reactor and allowing them to react.
